(19) United States
(12) Patent Application Publication
(10) Pub. No.: US 2002/0010503 A1
(43) Pub. Date: Jan. 24, 2002

(54) RADIOACTIVE STENT

(76) Inventor: RANDOLF VON OEPEN, HIRRLINGEN (DE)

Correspondence Address: NICOLA A PISANO, FISH & NEAVE, 1251 AVENUE OF THE AMERICAS, NEW YORK, NY, 10020, US

(21) Appl. No.: 09/303,849

(22) Filed: May 3, 1999

(30) Foreign Application Priority Data
May 5, 1998 (DE) 198 19 635.0

(51) Int. Cl. A61F 2/06; A61N 5/00

(52) U.S. Cl. 623/1.15; 600/3

(57) ABSTRACT

A stent for implanting in a body vessel has a stent element; and a plurality of receptors provided on the stent element for receiving medical substances.

### BACKGROUND OF THE INVENTION

The present invention relates to stents for implantation in blood vessels.

Stents have the objective to prevent a repeated narrowing or a repeated closure of the blood vessels after a dilation, in order to guarantee a good throughflow of blood. Stents can be used however not only in blood vessels, but also in other hollow vessels, such as for example urethria or bilducts.

Numerous studies showed that stents can substantially reduce the tendency of the vessels to reduce again or to close again. Despite this, during use of stents, in approximately 30% of all cases so-called residual eyes are produced, or in other words a new narrowing of the vessel in the region of the stent. This new narrowing can be caused for example by a proliferation of smooth muscle cells.

For this reason numerous efforts have been taken to reduce these residual eyes. For cancer therapy, it is known that a growth can be reduced or suppressed by a predetermined action of the cells with radioactivity. The cells during this treatment are deliberately damaged so as to suppress a further growth.

U.S. Pat. No. 5,722,984 discloses a preferably metallic stent which is radioactively marked. After the introduction in the sick vessel, weak radioactivity which is released over time counteracts a growth of the smooth muscle cells. The use of the radioactive stent is in practice, however, possible only with some difficulties. The half-lifetime of the product must be taken into consideration, which leads to substantial logistic problems. Radioactive stents can be used only within very short time, since otherwise an accurate dosage of the radioactivity can not be secured. Great logistic problems are also expected during transportation of the stent from a manufacturer to a user. Depending on selection of the radiation and level of dosing, safety measures must be taken during the transportation for reliable screening of the stents.

Regardless of the dosage and the utilized radiation, numerous safety precautions must be provided in individual catheter laboratories. In addition, such a treatment can be used only by protected customers, and thereby it is not accessible to a broad mass of practicing cardiologists.

### SUMMARY OF THE INVENTION

Accordingly, it is an object of the present of invention to provide a stent of the above mentioned type, which avoids the disadvantages of the prior art.

In keeping with these objects and with others which will become apparent hereinafter, one feature of present invention resides, briefly stated in a stent for implanting in a body vessel, which has receptors for receiving medical substances, in particular radioactively marked substances.

When the stent is designed in accordance with the present invention, it counteracts in particular growth of smooth muscle cells and therefore avoids the above mentioned disadvantages.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### DESCRIPTION OF PREFERRED EMBODIMENTS

In accordance with the present invention, a stent for implanting in a body vessel is proposed, which has receptors for receiving of medical substances, in particular radioactive marked substances.

The stent in accordance with the present invention uncouples the process of stenting and the process of introduction of the radio activity or another medical substance. A stent under a protective procedure of cardiologists is placed in a sick vessel via a suitable device, for example by a balloon catheter, at the location to be treated. The stent is provided with special receptors, which for example have a high affinity to predetermined substances. These receptors adhere to the surface of the stent and provide the desired coupling of the substances which are characterized by a high affinity to the receptors located on the surface of the stent.

Such a substance, provided with a high affinity to the receptors, can be for example enriched with radioactivity. Thereby it is guaranteed that a stent, provided with the corresponding receptors, can receive an exactly definite quantity of the substances which are to be introduced in the body of the patient, for example intravenously, and thereby an exact dosage of the radioactivity can be adjusted.

The process of binding a radioactive substance can run in two or more stages. The stent makes possible, by the receptors specifically anchored on the surface, to bind special substances. This substance must not be primarily a substance in which the radioactivity is coupled, but instead it also can be a substance which in turn has a great affinity to a radioactive substance. The uncoupling of the process between stenting and activation of the medical efficiency of the stent enables a practicing cardiologist to determine individuality whether such therapy is desirable and what dosage is to be selected for the corresponding patients.

The activation of the stent can be performed directly by a protective personnel in a catheter laboratory. On the other hand, preparation is performed in departments, in which the technology with the corresponding safety measures for radiation therapy are available.

When the stent is provided with different receptors or builds the deposited substance on the receptors over a certain time, the procedure of the activation can be repeated when necessary.

The receptors can be formed as a layer applied on the stent. Further embodiments of the inventive stent can include a sleeve and/or an envelope. The selection of the stent material is not limited to a medical high grade steel. All materials which have a sufficiently high acceptance in an organism can be utilized.

It must be guaranteed that both the receptors and the substances introduced in the body of patients have a sufficient body compatibility.

The technology of arranging the receptors on the surface of the stent is not limited in any case to the addition of radioactive substances. The receptors can be also formed so that a high affinity to medications is provided, which for example must be discharged in a dosed manner into the body over a long time. The placements of such medications on the receptors corresponds to the placement up of radio active substances as described herein above.

The invention is not limited to coupling of an individual receptor. Also a plurality of different receptors can be bonded to the surface, to perform a treatment with different medications. The receptors can be contained in the material of the stent. This is provided in particular when a rational manufacture is desired. In order to make possible exact dosing of the medical substances or to prevent overdosing, the layer and/or the sleeve and/or the coating can be dissolved after a certain time. For the same reasons, the receptors can be formed so that they loose their infinity for certain substances after a predetermined time.

The receptors can be applied by a catheter afterwards, so that the stent can be made therapeutically efficient in the body, individually depending on the desired therapy.

It will be understood that each of the elements described above, or two or more together, may also find a useful application in other types of constructions differing from the types described above.

While the invention has been illustrated and described as embodied in radioactive stent, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A stent for implanting in a body vessel, comprising a stent element; and a plurality of receptors provided on said stent element for receiving medical substances.

2. A stent as defined in claim 1, wherein said receptors are provided for receiving radioactive marked substances.

3. A stent as defined in claim 2, wherein said receptors have a high affinity for the radioactive marked substances.

4. A stent as defined in claim 1, wherein said receptors have a high affinity for chemical substances.

5. A stent as defined in claim 1, wherein said receptors are arranged in a layer and provided for medical substances.

6. A stent as defined in claim 1; and further comprising a sleeve provided with said receptors for medical substances.

7. A stent as defined in claim 1; and further comprising a coating provided with said receptors for medical substances.

8. A stent as defined in claim 5, wherein said layer of receptors is formed so as to dissolve after a certain time.

9. A stent as defined in claim 6, wherein said sleeve with said receptors is formed so that it dissolves after a certain time.

10. A stent as defined in claim 7, wherein said coating with said receptors is formed so that it releases after a predetermined time.

11. A stent as defined in claim 1, wherein said receptors are formed so that they loose their affinity for said substances after a predetermined time.

12. A stent as defined in claim 1, wherein said receptors are formed so that they are mountable by a catheter.

13. A stent as defined in claim 1, wherein said receptors are contained in a material of said stent element.

14. A stent as defined in claim 1, wherein said substances have a high affinity for further substances.

15. A stent as defined in claim 14, wherein said substances have a high affinity for radioactive substances.

16. A stent as defined in claim 1, wherein said substances are decomposable by a body after a predetermined time.
